(19) United States
(12) Patent Application Publication
(10) Pub. No.: US 2013/0190895 A1
(43) Pub. Date: Jul. 25, 2013

(54) SLEEVE HAVING A REINFORCEMENT LAYER

(71) Applicant: Ossur hf, Reykjavik (IS)

(72) Inventor: Hronn KRISTINSDOTTIR, Reykjavik (IS)

(73) Assignee: Ossur hf, Reykjavik (IS)

(21) Appl. No.: 13/743,542

(22) Filed: Jan. 17, 2013

Related U.S. Application Data
(60) Provisional application No. 61/589,931, filed on Jan. 24, 2012.

(51) Int. Cl. A61F 2/78

(52) U.S. Cl. CPC A61F 2/7812
USPC 623/36

(57) ABSTRACT

A sleeve has interior and exterior surfaces, a proximal opening in a proximal area, and a distal area, and is adapted to be worn on a residual limb and at least partially over a definitive socket. The sleeve includes a fabric layer continuously forming the exterior surface of the sleeve. A polymeric layer is bonded to the fabric layer and forms the interior surface of the sleeve. A reinforcement layer is arranged in a localized area of the sleeve between the proximal and distal areas of the sleeve.

### FIELD OF ART

The disclosure relates to the field of prosthetic devices, and more particularly to a sleeve including a reinforcement layer for use with a socket.

### BACKGROUND

In certain applications, an amputee wears a liner over a residual limb as an interface between a definitive socket and the residual limb. The socket is attached to the residual limb and liner by a suspension mechanism. Besides the liner, a sleeve is commonly worn over the socket and liner combination for additional suspension of the prosthetic limb by preventing air from entering or exiting the proximal end of the socket. The sleeve extends distally over the brims of the socket and elastically conforms to the residual limb and socket to form a seal between the socket and the liner.

Because of the desire to produce a tight fluid seal with the liner and socket, the sleeve necessarily closely conforms to the surface contours of the residual limb, liner, and socket. This characteristic of the sleeve results in added stress to areas of the sleeve which come into contact with the brims of the socket. The interior layer of the sleeve is especially susceptible to being deformed by the socket brims.

Over the knee area, the sleeve stretches over the knee. Some sleeves may include a greater thickness around the socket brims. This construction may avoid early wear and tear of the sleeve, but also may create resistance at the knee and add to material costs of and weight of the sleeve. This reduces both comfort and flexibility of the sleeve.

Therefore, there is a need in the prosthetic art to provide sleeves with improved durability while maintaining the comfort and flexibility to adapt to the needs of each user.

### SUMMARY

The challenges of known prosthetic devices are addressed under embodiments described providing a sleeve having a reinforcement layer. Adding a reinforcement layer or element to the sleeve provides more support to the device in localized areas to strengthen the sleeve against stresses from contact with the brims of a socket. The reinforcement layer allows for better durability and comfort.

Under an embodiment of the device, a tubular sleeve has interior and exterior surfaces, a proximal opening in a proximal area, and a distal area, and is adapted to be worn on a residual limb. The sleeve includes a fabric layer, a polymeric layer, and a reinforcement layer. The fabric layer has interior and exterior surfaces and continuously forms the exterior surface of the sleeve. The polymeric layer is bonded to the fabric layer and forms the interior surface of the sleeve. The reinforcement layer is arranged in a localized area of the sleeve with the localized area being between a proximal area and a distal area of the sleeve.

In an embodiment of the sleeve, the reinforcement layer may extend radially around the entire circumference of the sleeve. The reinforcement layer may have limited or no elasticity in the longitudinal direction of the sleeve. The thickness may generally be consistent across the length of the sleeve, and the reinforcement layer does not impede flexibility or contribute to adding weight to the sleeve.

In another embodiment of the sleeve, the reinforcement layer may extend radially around most of the circumference of the sleeve, and the reinforcement layer may be elastic in the longitudinal direction of the sleeve.

A method involves placing the sleeve over a definitive socket and aligning a localized area of the sleeve with a brim of the socket. The sleeve may have an area of curvature corresponding to the reinforcement layer that accommodates the knee joint.

### BRIEF DESCRIPTION OF THE DRAWINGS

The sleeve is described referring to the accompanying drawings which show preferred embodiments according to the device described. The sleeve as disclosed in the accompanying drawings is illustrated for example only. The elements and combinations of elements described below and illustrated in the drawings can be arranged and organized differently to result in embodiments still within the spirit and scope of the device described.

FIG. 1 shows an anterior view of an embodiment of the sleeve.

FIG. 2 shows an anterior view of an embodiment of the sleeve worn on a residual limb with a socket.

FIG. 3 shows a view of a second embodiment of the sleeve.

FIG. 4 shows a cross-section of an embodiment of the sleeve.

### DETAILED DESCRIPTION OF VARIOUS EMBODIMENTS

A better understanding of different embodiments of the sleeve may be had from the following description read with the accompanying drawings in which like reference characters refer to like elements.

While the disclosure is susceptible to various modifications and alternative constructions, certain illustrative embodiments are in the drawings and will be described below. It should be understood, however, there is no intention to limit the disclosure to the specific embodiments disclosed, but on the contrary, the intention covers all modifications, alternative constructions, combinations, and equivalents falling within the spirit and scope of the disclosure and defined by the appended claims.

It will be understood that, unless a term is expressly defined in this disclosure to possess a described meaning, there is no intent to limit the meaning of such term, either expressly or indirectly, beyond its plain or ordinary meaning.

Any element in a claim that does not explicitly state “means for” performing a specified function, or “step for” performing a specific function is not to be interpreted as a “means” or “step” clause as specified in 35 U.S.C. §112, paragraph 6.

The anatomical terms described are not intended to detract from the normal understanding of such terms as readily understood by one of ordinary skill in the art of prosthetics. The term “distal” is used to denote the portion or end of a limb farthest from the central portion of the body. The term distal is the opposite of “proximal” which is used to denote that the end or portion of the limb is nearer to the central portion of the body.

Some components described share similarities to components in U.S. Pat. No. 6,706,364 granted Mar. 16, 2004, incorporated by reference and belonging to the assignee of this disclosure.

The embodiments described may be used with a socket as described in U.S. Pat. No. 6,589,289, granted Jul. 8, 2003 granted Mar. 16, 2004, incorporated by reference and belonging to the assignee of this disclosure.

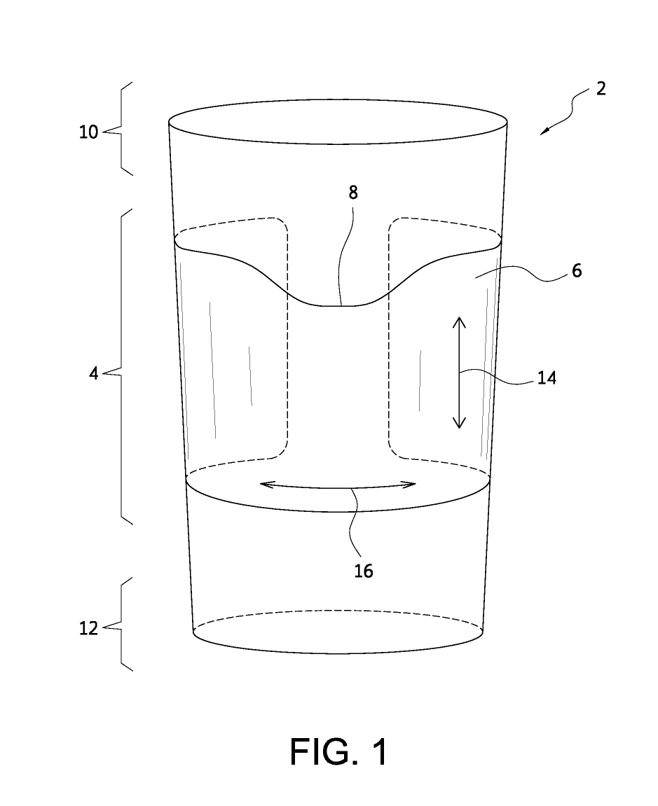

In an exemplary embodiment of the prosthetic device, FIG. 1 shows a sleeve 2 from the anterior side for transtibial (TT) amputees. When the sleeve 2 is used with a socket, an interior radial area of the sleeve 2 comes into direct contact with the brim of the socket. A localized area 4 is present around the radial contact area between a proximal area 10 and a distal area 12. The reinforcement layer 6 is in the localized area 4 of the sleeve which extends proximally and distally from the radial contact area to provide a more durable area of the sleeve since the radial area is more susceptible to tearing due to the shearing and frictional forces caused by contact with the socket and socket brim during gait. The localized area 4 may have a longitudinal length such that if the sleeve 2 and socket move regarding each other during ambulation, the changing radial contact area is within the localized area 4.

The reinforcement layer is provided at least on the anterior side of the sleeve 2 and may extend radially around the circumference of the sleeve 2. The reinforcement layer 6 may extend symmetrically around sleeve from the center point of the anterior side to between approximately 50% to 75% of the total circumference of the sleeve. For TT amputees, the reinforcement layer 6 preferably does not extend completely around the circumference of the sleeve to allow the knee to have a greater range of motion. Having the reinforcement layer present around the entire circumference of a sleeve 2 designed for a TT amputee would undesirably restrict the motion of the wearer's knee.

Besides limiting the circumference of the reinforcement layer 6 for better knee flexion, the reinforcement layer 6 is formed from a textile material having a matrix of fibers which may be elastic in a longitudinal direction 14. The fibers of the textile material can have limited elasticity in a radial direction 16 to provide support while allowing for easy donning and doffing of the sleeve 2 and good proprioception. The reinforcement layer 6 also has an area of curvature 8 having a lesser longitudinal length on the anterior side to accommodate the knee of the wearer. The reinforcement layer 6 is preferably present directly below the knee area of the sleeve 2.

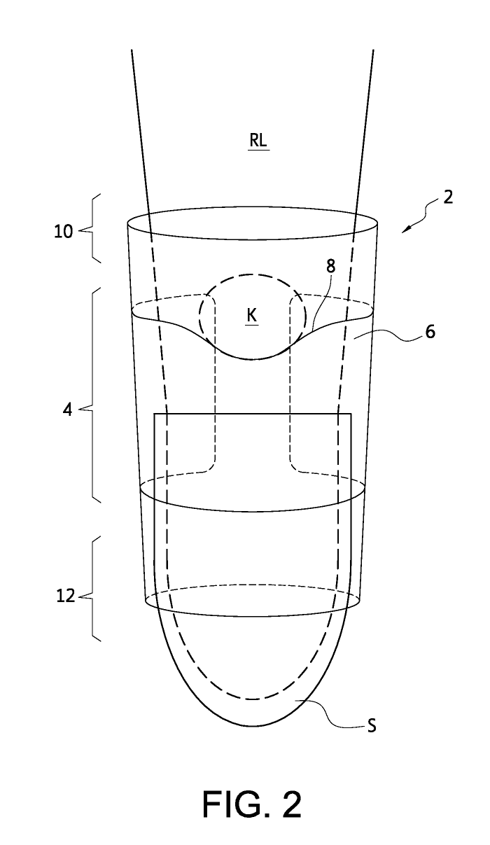

FIG. 2 illustrates sleeve 2 worn on a residual limb RL with a socket S. The residual limb RL has a knee joint K, and the sleeve 2 is oriented so the knee K is accommodated by the area of curvature 8. The sleeve 2 is worn so the brim of the socket S is within the localized area 4.

Due to the different needs of TT amputees and transfemoral (TF) amputees, the placement and properties of the reinforcement layer varies for sleeves for TT amputees and TF amputees.

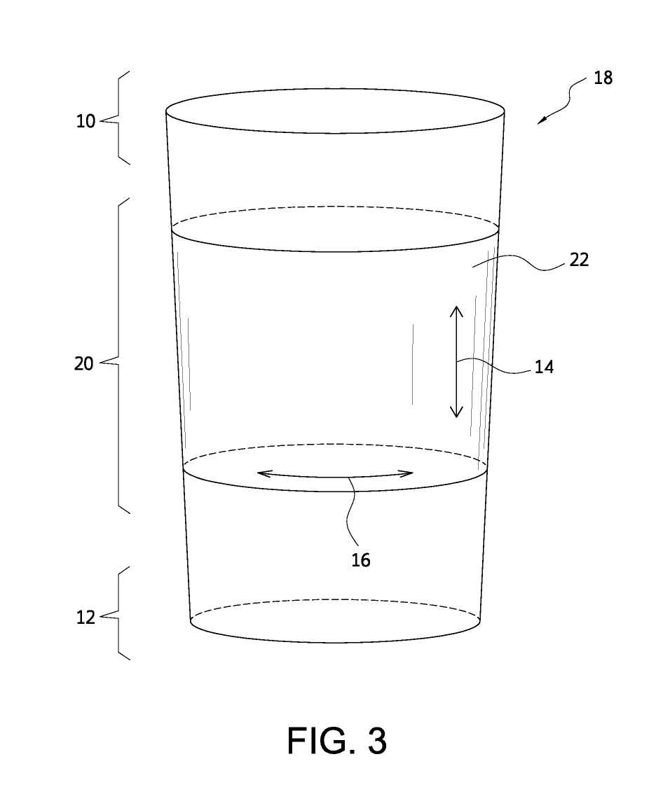

FIG. 3 shows an embodiment of a sleeve 18 for TF amputees. Similar to the TT sleeve 2, the TF sleeve 18 has a localized area 20 in which the reinforcement layer 22 is located, a proximal area 10, and a distal area 12. Since the TF sleeve 18 need not accommodate a flexing joint, the reinforcement layer 22 may be formed from a textile material having a matrix of fibers which has limited elasticity or no elasticity in the longitudinal direction 14 and extends around the entire circumference of the TF sleeve 18. Like the TT sleeve 2, the textile material of the reinforcement layer 22 can have some elasticity in the radial direction 16 to allow for easier donning and doffing of the sleeve while providing sufficient support to the sleeve to increase resistance to the shearing forces of the socket brim.

The sleeve 18 may be worn on a residual limb in a similar manner to the exemplary embodiment in FIG. 2.

As shown in FIGS. 1-3, the reinforcement layer 6 may have an overall rectangular shape for consistent support across the localized area 4. The reinforcement layer 6 may also have different shapes to provide support to only targeted areas of the sleeve. The reinforcement layer 6 can have the same dimensions as the localized area 4.

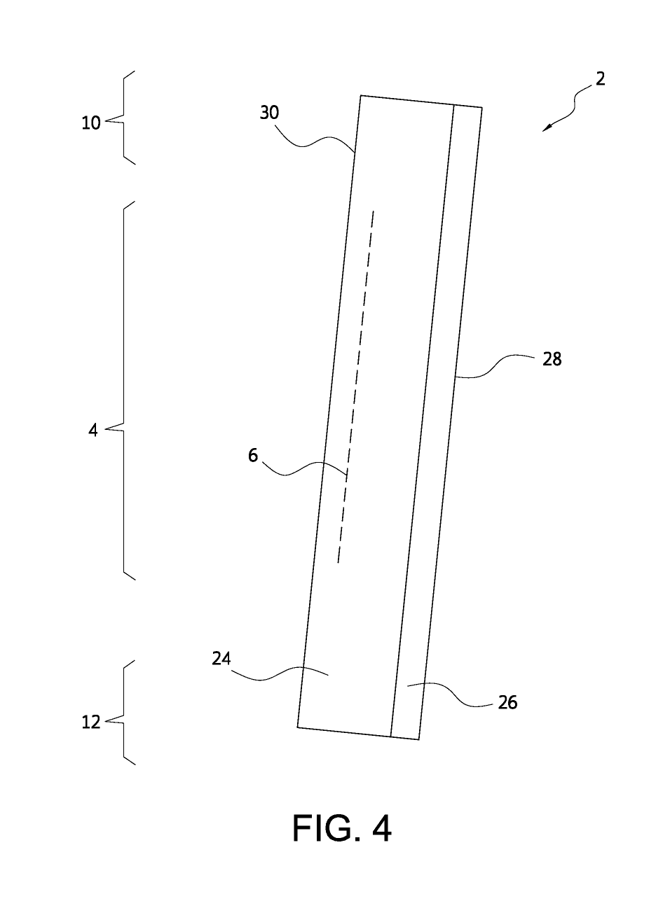

FIG. 4 shows a cross-sectional portion of an embodiment of a sleeve. The features shown in FIG. 4 are described regarding the TT sleeve 2 but apply also to the TF sleeve 18. The sleeve 2 is formed from a layer of polymeric material 24 such as a silicone elastomer bonded to a fabric layer 26. The silicone elastomer 24 is on the interior side of the sleeve 2 and forms an interface with the skin of the residual limb or a liner when worn. In this embodiment, the fabric layer 26 defines the exterior surface 28 of the sleeve, whereas the silicone elastomer 24 defines the interior surface 30 of the sleeve.

The reinforcement layer may be placed in a variety of locations within the thickness of the sleeve 2. As shown in FIG. 3, the reinforcement layer 6 is preferably embedded within the silicone elastomer layer 24. The reinforcement layer 6 may be embedded nearer to the interior surface 30 of the sleeve 2 since the material closer to the interior 30 of the sleeve 2 is subject to greater shearing stresses from the socket brims. For example, the reinforcement layer is embedded within the polymeric layer between a middle point in the thickness of the polymeric layer and the interior surface of the sleeve.

The reinforcement layer 6 is formed from a textile material with a sufficiently high tensile strength to withstand the pressure and forces applied to it by the elastic properties of the fabric layer and the silicone layer and the shearing force of the socket brim. It is preferable that the textile material is elastic in certain directions. However, the textile material the modulus of elasticity should not be too high. A balance between radial elasticity of the reinforcement layer and the tensile strength should be achieved to provide easy donning and doffing and good proprioception.

The textile material may be formed from a circular knit textile or a flat knit textile. Over one piece of textile may form the reinforcement layer 6 to provide different reinforcing effects. Any material that is more rigid than the polymeric layer 24 and has the desired characteristics described may be used to form the reinforcement layer 6 such as a polymeric mesh, knitted glass fiber, or Nylon.

The reinforcement layer 6 may be embedded between the silicone elastomer layer 24 and the fabric layer 26 for better sealing between the residual limb, the sleeve, and the socket. The reinforcement layer 6 may also be embedded between two silicone elastomer layers as described in U.S. Pat. No. 6,136,039, granted Oct. 24, 2000, incorporated by reference and belonging to the assignee of this disclosure. The reinforcement layer 6 may also be attached to the outer fabric layer of the sleeve using stitching or an adhesive.

Over one reinforcement layer 6 may be used with the embodiments described. The multiple reinforcement layers may be elastic or inelastic in different directions to combine to produce a more specific reinforcing effect. One reinforcement layer may be elastic in a radial direction and a second reinforcement layer is inelastic in the longitudinal direction.

The silicone elastomer layer 24 preferably has a consistent thickness throughout the sleeve to enable the sleeve to better mold to the surface contours of the residual limb and/or liner and socket. The silicone elastomer layer 24 may also have a greater thickness within the localized area 4 for a more durable reinforced area around the socket brim.

The fabric layer 26 of the sleeve is preferably elastic or elasticized. The fabric layer 26 may include one or more fabric sections including fabric sections having different elastic stiffnesses as described in U.S. Pat. No. 6,592,539, granted Jul. 15, 2003, incorporated by reference and belonging to the assignee of this disclosure.

The embodiments described may also be used with the peripheral profile features of the sleeve described in U.S. Pat. No. 7,169,189, ranted Jan. 30, 2007, incorporated by reference and belonging to the assignee of this disclosure to provide improved conformability, pressure distribution and stretchability to the sleeve.

The silicone elastomer layer 24 of the sleeve may have additional supplements such as petroleum jelly and aloe vera to improve skin care and comfort also described in U.S. Pat. No. 7,169,189.

From the features of the sleeve described above, the textile reinforcement layer provides reinforcement to select areas of a sleeve susceptible to shearing stress from the brim of a socket. Adding the textile reinforcement layer to the sleeve increases the overall durability of the sleeve while maintaining the sleeve's ability to conform to the contours of the residual limb and socket to create strong suspension of the prosthesis.

The reinforcement layer as described may be used similarly in a liner for reinforcement of select areas. Like a sleeve, a liner may have a polymeric interior layer bonded to a fabric outer layer where the polymeric layer forms an interface with the skin of the residual limb. The reinforcement layer or layers may be used in specific areas of the liner and provided in various locations within the thickness of the liner. The reinforcement layer or layers used in the liner may be formed from the material described with respect to the reinforcement layer.

The sleeve described may also be an orthotic sleeve to provide support to an area of a limb such as support and reinforce muscles or joints. The localized area may be aligned and oriented so the selected muscle or joints are within the localized area having a reinforcement layer.

While the foregoing embodiments have been described and shown, alternatives and modifications of these embodiments, such as those suggested by others, may be made to fall within the scope of the invention. The principles described may be extended to other types of prosthetic or orthopedic devices.

1. A tubular sleeve defining interior and exterior surfaces, a proximal opening in a proximal area, and a distal area, and the sleeve adapted to be worn on a residual limb and at least partially over a definitive socket, comprising:
a fabric layer defining inner and outer sides, and continuously forming the exterior surface of the sleeve;
a polymeric layer secured to the inner side of the fabric layer and forming the interior surface of the sleeve; and
a reinforcement layer arranged in a localized area of the sleeve outside of and between the proximal and distal areas of the sleeve.

2. The sleeve according to claim 1, wherein the reinforcement layer is embedded within the polymeric layer.

3. The sleeve according to claim 1, wherein the reinforcement layer is embedded within the polymeric layer between a middle point of a thickness of the polymeric layer and the interior surface of the sleeve.

4. The sleeve according to claim 1, wherein the reinforcement layer is bonded to the polymeric layer.

5. The sleeve according to claim 1, wherein the reinforcement layer extends radially on an anterior side of the sleeve.

6. The sleeve according to claim 1, wherein the reinforcement layer extends radially on anterior and posterior sides of the sleeve.

7. The sleeve according to claim 1, wherein the reinforcement layer is formed from inelastic textile material.

8. The sleeve according to claim 1, wherein the reinforcement layer is formed from elastic textile material.

9. The sleeve according to claim 8, wherein the elastic textile material stretches longitudinally.

10. The sleeve according to claim 1, wherein the reinforcement layer extends radially completely around the circumference of the sleeve.

11. The sleeve according to claim 1, wherein the fabric layer is formed from elasticized fabric.

12. The sleeve according to claim 1, wherein the reinforcement layer is formed from a textile material having a matrix of fibers elastic in a longitudinal direction, the fibers of the textile material having limited elasticity in a radial direction, the reinforcement layer defining an area of curvature having a lesser longitudinal length on an anterior side of the sleeve.

13. The sleeve according to claim 1, wherein the reinforcement layer has an area of curvature around a radial center of an anterior side of the sleeve.

14. The sleeve according to claim 1, wherein the reinforcement layer is formed from a textile material including a matrix of fibers having limited or no elasticity in a longitudinal direction of the sleeve and extending around an entire circumference of the sleeve.

15. The sleeve according to claim 1, wherein the reinforcement layer extends symmetrically around the sleeve from a center point of an anterior side to between approximately 50% to 75% of a total circumference of the sleeve.

16. A sleeve having interior and exterior surfaces, a proximal opening in a proximal area, and a distal area, and adapted to be worn on a residual limb and at least partially over a definitive socket, comprising:
a fabric layer defining inner and outer sides, the fabric layer continuously forming the exterior surface of the sleeve and having at least one fabric piece;
inner and outer polymeric layers secured to each other, the inner polymeric layer forming the interior surface of the sleeve; and
at least one reinforcement layer arranged in a localized area of the sleeve, the localized area in an area between the proximal and distal areas of the sleeve, the reinforcement layer extending radially around a circumference of the sleeve;
wherein the reinforcement layer is embedded within the polymeric layer between a middle point of a thickness of the polymeric layer and the interior surface of the sleeve.

17. The sleeve according to claim 16, wherein the reinforcement layer is formed from a textile material including a matrix of fibers having limited or no elasticity in a longitudinal direction of the sleeve.

18. The sleeve according to claim 16, wherein the reinforcement layer has an area of curvature around a radial center of an anterior side of the sleeve.

19. A method of donning a sleeve having a reinforcement layer arranged in a localized area of the sleeve onto a definitive socket, comprising the steps of:
donning the sleeve on a residual limb through a proximal opening of the sleeve;
aligning the localized area of the sleeve with the brim of the socket;
radially securing the sleeve to the socket and maintaining the localized area in place during ambulation.

20. The method according to claim 19, further comprising the step of aligning the localized area to a socket brim so that the socket brim remains within the localized area during ambulation.
